(19) United States
(12) Patent Application Publication
(10) Pub. No.: US 2021/0196946 A1
(43) Pub. Date: Jul. 1, 2021

(54) SPINE POSTURE CORRECTION DEVICE

(71) Applicant: RC Technologies, LLC, Albuquerque, NM (US)

(72) Inventor: Raciel Alexis Castillo, Albuquerque, NM (US)

(21) Appl. No.: 17/135,834

(22) Filed: Dec. 28, 2020

Related U.S. Application Data
(60) Provisional application No. 62/954,443, filed on Dec. 28, 2019.

(51) Int. Cl. A61N 1/04; A61H 39/00; A61H 39/04

(52) U.S. Cl. CPC A61N 1/0456; A61H 39/002; A61H 39/04; A61H 2205/081; A61H 2201/1623; A61H 2201/10; A61H 2201/0119; A61H 2201/5097

(57) ABSTRACT

A method, system, and apparatus for posture correction comprises a backplate, at least two pressure point knobs affixed to the backplate, and a strap operably connected to the backplate. Mounting slots are formed in the backplate with mounting bars bisecting the mounting slots, the mounting slots and mounting bars forming a strap mounting point through which the strap can be threaded, and at least two pressure point mounting slots are formed on the backplate, wherein each of the at least two pressure point knobs can be mounted to the backplate via the at least two pressure point mounting slots.

### CROSS REFERENCE TO RELATED APPLICATIONS

This patent application claims the priority and benefit under 35 U.S.C. § 119(e) of U.S. Provisional Patent Application Ser. No. 62/954,443, filed Dec. 28, 2019, entitled “The Natural Spine Posture Correction Device.” U.S. Provisional Patent Application Ser. No. 62/954,443 is herein incorporated by reference in its entirety.

### TECHNICAL FIELD

Embodiments are generally related to orthopedic devices. Embodiments are further related to posture correcting devices. Embodiments are also related to methods and systems for naturally adjusting and/or correcting spine posture. Embodiments are also related to methods and systems for relieving pain associated with poor posture. Embodiments are additionally related to a systems and apparatuses for natural spine posture correction.

### BACKGROUND

Slouching while seated causes tremendous strain on the spine. Over time this strain can result in acute or chronic back pain. This fundamentally results because slouching creates a weight distribution on back muscles that forces them to overcompensate, pulling the spine's vertebrae in opposing directions, which can lead to injury.

Bad posture is an incredibly common problem. Up to 80% of the population will in their lifetime, suffer from bad posture. Numerous attempts to address this endemic problem exist. Many such solutions include orthopedic devices that force the user's spine into proper alignment. These solutions may offer temporary corrective posture but do not help teach the user how to sit correctly.

Other currently available solutions can exacerbate problems, causing unnatural strain by pushing the user's spine into position. Such solutions may create a dull sensation in and or around the erector spinae muscles and spine. Prolonged use can be uncomfortable and ineffective. In some cases, such devices may even cause injury.

Accordingly, there is a need in the art for natural spine posture correction devices as disclosed in the embodiments detailed herein.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide methods and systems for posture correction.

It is another aspect of the disclosed embodiments to provide systems and apparatuses for naturally correcting spine alignment.

It is another aspect of the disclosed embodiments to provide methods, systems, and apparatuses for naturally correcting sitting posture.

The aforementioned aspects and other objectives and advantages can now be achieved as described herein. In one embodiment, a system and/or apparatus for posture correction comprises a backplate, at least two pressure point knobs affixed to the backplate, and a strap operably connected to the backplate.

In an embodiment, the system comprises at least one mounting slot formed in the backplate and at least one mounting bar bisecting the mounting slot, the mounting slot and mounting bar forming a strap mounting point through which the strap can be threaded.

In an embodiment, the system comprises at least two pressure point mounting slots formed on the backplate, wherein each of the at least two pressure point knobs can be mounted to the backplate via the at least two pressure point mounting slots. In certain embodiments, the at least two pressure point mounting slots comprise straight slots configured at a substantially matching elevation on the backplate. In certain embodiments, the at least two pressure point mounting slots comprise straight slots and are oriented to be parallel and are configured at a substantially matching elevation on the backplate. In certain embodiments, the at least two pressure point mounting slots comprise cross shaped slots and are configured at a substantially matching elevation on the backplate.

In another embodiment a posture corrective system comprises a backplate, at least two pressure point knobs affixed to the backplate, at least one controller formed on the backplate configured to control a location of the at least two pressure point knobs, at least one battery, and a strap operably connected to the backplate. In an embodiment, the system comprises at least one strap adjusting assembly operably connected to the at least one controller and configured to adjust a location of the backplate on the strap. In an embodiment, the system comprises at least two knob adjusting assemblies operably connected to the at least one controller and configured to adjust the respective location of the at least two pressure point knobs.

### BRIEF DESCRIPTION OF THE DRAWINGS

The accompanying figures, in which like reference numerals refer to identical or functionally-similar elements throughout the separate views and which are incorporated in, and form a part of, the specification, further illustrate the embodiments and, together with the detailed description, serve to explain the embodiments disclosed herein.

FIG. 1 depicts a posture corrective system, in accordance with the disclosed embodiments;

FIG. 2A depicts top view of a backplate, in accordance with the disclosed embodiments;

FIG. 2B depicts another embodiment of a backplate, in accordance with the disclosed embodiments;

FIG. 2C depicts another embodiment of a backplate, in accordance with the disclosed embodiments;

FIG. 2D depicts another embodiment of a backplate, in accordance with the disclosed embodiments;

FIG. 3A depicts a perspective view of a pressure point knob, in accordance with the disclosed embodiments;

FIG. 3B depicts another view of a pressure point knob, in accordance with the disclosed embodiments;

FIG. 3C depicts a side view of a pressure point knob, in accordance with the disclosed embodiments;

FIG. 4 depicts a posture corrective system installed on a seat, in accordance with the disclosed embodiments;

FIG. 5A depicts a remotely controllable posture corrective system, in accordance with the disclosed embodiments;

FIG. 5B depicts a backplate associated with a remotely controllable posture corrective system, in accordance with the disclosed embodiments; and

FIG. 6 depicts an embodiment of a strap, in accordance with the disclosed embodiments.

### DETAILED DESCRIPTION

The particular values and configurations discussed in the following non-limiting examples can be varied, and are cited merely to illustrate one or more embodiments and are not intended to limit the scope thereof.

Example embodiments will now be described more fully hereinafter with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. In addition, the term “based on” may be understood as not necessarily intended to convey an exclusive set of factors and may, instead, allow for existence of additional factors not necessarily expressly described, again, depending at least in part on context.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The embodiments disclosed herein relate to a posture corrective device that does not force the spine into proper alignment, but instead is configured for the user to sit up against pressure points to achieve natural alignment of the spine while simultaneously relaxing overworked or stressed erector spinae muscles. The disclosed embodiments not only help to correct poor posture, but also provide immediate pain relief.

In certain embodiments, a system as disclosed herein can include two pressure points mounted to a backplate which can be affixed to any sort of seating accommodation, backpack, pillow, or the like. The pressure points press into the erector spinae muscles pushing forward at key points along the spine (e.g., cervical, thoracic, and/or lumbar regions), allowing overworked muscles holding the spine upright, to relax. By relaxing the muscles, the spine moves into its naturally curved position. It should be appreciated that, in other embodiments, the system can further be used on, or in association with, other muscle groups without departing from the scope of this disclosure.

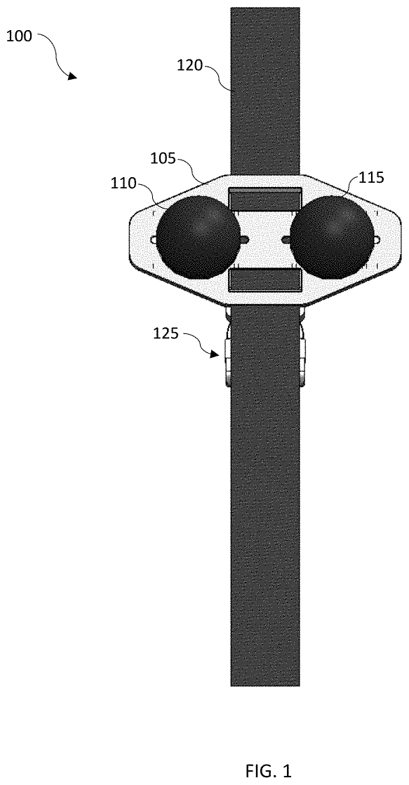

FIG. 1, illustrates a posture corrective system 100 in accordance with the disclosed embodiments. The posture corrective system 100 generally comprises a backplate 105, a first pressure point knob 110 and a second pressure point knob 115 which can be affixed to the backplate, the pressure point knobs being spaced to naturally engage the spine, and a strap 120 operably connected to the backplate. The strap 120 can have two loose ends each of which can be affixed to the male and female sides of a buckle 125, such that when the male and female sides of the buckle 125 are connected the strap forms a loop.

It should be appreciated that the relative size of the posture corrective system 100, and componentry thereof, can be adjusted according to the application of the system. For example, in certain embodiments, the system can include a generally larger backplate 105 and generally larger pressure point knobs 110 and 115. In other embodiments, configured for children, the system 100 can include a generally smaller backplate 105 and generally smaller pressure point knobs 110 and 115 sized to fit the anatomy of a child. In other embodiments, pressure point knobs can come in multiple sizes so that the system can be tailored to the current user, be that an adult, adolescent or child, male, female, etc.

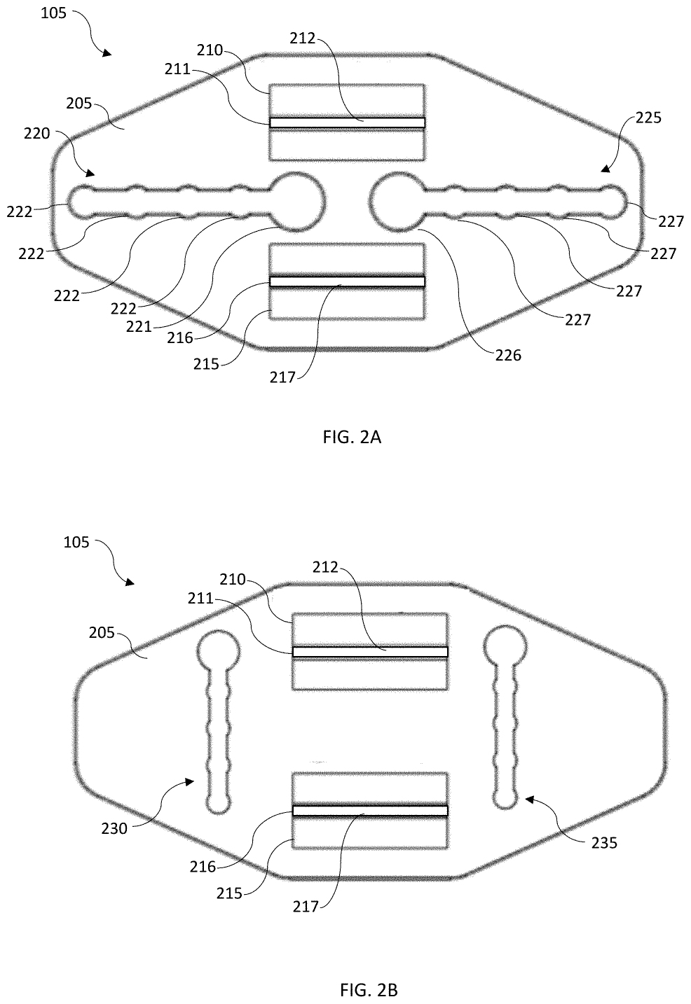

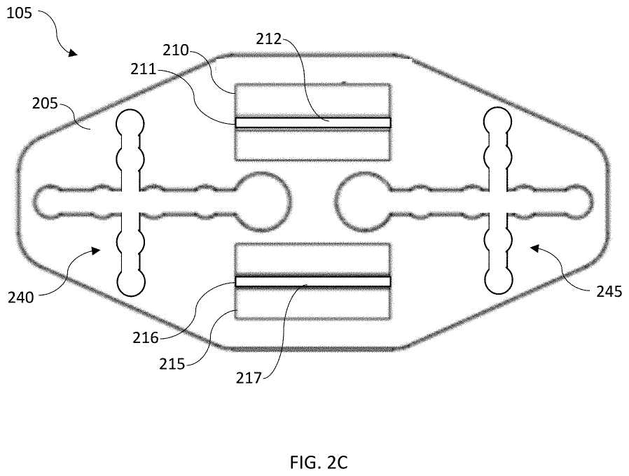

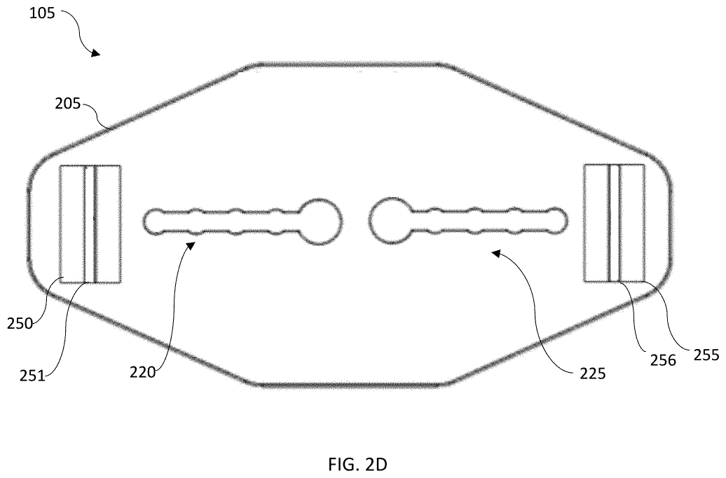

FIG. 2A illustrates an embodiment of the backplate 105 of the posture corrective system 100. The backplate 105 can comprise a substantially flat substrate 205. In certain embodiments, the substrate can be metal, plastic, rubber, or any other such rigid substrate. The substrate 205 is illustrated with a hexagon in FIG. 2A, but in other embodiments, other substrate shapes may be used without departing from the scope of the disclosure. However, it may be advantageous, in certain embodiments, for the width of the substrate 205 to be greater than the height, in order to accommodate multiple mounting positions of the pressure point knobs as further detailed herein.

The backplate 105 further comprises at least one, and in some embodiments, multiple mounting slots. In FIG. 2A a first mounting slot 210 and a second mounting slot 215 are illustrated. The mounting slots 210 and 215 are configured on the backplate 105 to be substantially in alignment. This ensure the mounting slots serve as attachment points for the strap 120.

Each of the mounting slots can include a mounting bar formed across the opening of the respective mounting slot. In FIG. 2A, mounting bar 211 is formed across mounting slot 210 and mounting bar 216 is formed across mounting slot 215. The mounting slots 210 and 215 and mounting bars 211 and 216 form strap mounting points through which the strap 120 can be threaded.

The top of mounting bar 211 can include a raised ridge 212 and the top of mounting bar 216 can include raised ridge 217. Raised ridge 212 and raised ridge 217 can be configured to facilitate engagement of the mounting strap in place. Raised ridge 212 and raised ridge 217 can comprise a curved hard surface or can be a semi-soft curved rubber surface, configured to provide friction against the strap 120. Slack in the strap 120 at the mounting slots allows the location of backplate 105 to be adjusted along the strap 120.

FIG. 2A illustrates first pressure point mounting slot 220 and second pressure point mounting slot 225 formed on the backplate 105. The two pressure point knobs can be mounted to the backplate 105 via the first pressure point mounting slot 220 and second pressure point mounting slot 225. As illustrated in FIG. 2, the first pressure point mounting slot 220 and second pressure point mounting slot 225 can comprise substantially straight slots configured at substantially matching elevations on the backplate substrate 205.

The first pressure point mounting slot 220 can further comprise a larger aperture 221 configured on the inner pointing end of the slot, and can comprise a series of additional smaller aperture cutouts 222 configured periodically along the length of the slot. The larger aperture 221 is configured to allow a peg and post associated with one of the pressure point knobs to be inserted into the first pressure point mounting slot 220. The pressure point knob can then slide into position along the first pressure point mounting slot 220 in one of the smaller aperture cutouts 222.

Likewise, the second pressure point mounting slot 225 can further comprise a larger aperture 226 configured on the inner pointing end of the slot, and can comprise a series of additional smaller aperture cutouts 227 configured periodically along the length of the slot. The larger aperture 226 is configured to allow a peg and post associated with one of the pressure point knobs to be inserted into the second pressure point mounting slot 225. The pressure point knob can then slide into position along the second pressure point mounting slot 225 in one of the smaller aperture cutouts 227.

It should be noted that the smaller aperture cutouts 222 and smaller aperture cutouts 227 allow the relative locations of the first pressure point knob 110 and second pressure point knob 115 to be adjusted with respect to one another as required to provide the desired posture corrective pressure and pain relief.

FIG. 2B illustrates backplate 105 with first pressure point mounting slot 230 and second pressure point mounting slot 235 oriented vertically on the backplate 105. In such an embodiment, the first pressure point mounting slot 230 and second pressure point mounting slot 235 can be substantially identical to the first pressure point mounting slot 220 and second pressure point mounting slot 225, except that the first pressure point mounting slot 230 and second pressure point mounting slot 235 are oriented to be vertical and parallel and are configured at a substantially matching elevation on the backplate to provide vertically adjustable locations for the pressure point knobs. In certain cases, it may be advantageous for the pressure point knobs to be vertically offset to provide maximum pain relief.

FIG. 2C illustrates backplate 105 with first pressure point mounting slot cross 240 and second pressure point mounting slot cross 245 oriented on the backplate 105. In such an embodiment, the first pressure point mounting slot cross 240 and second pressure point mounting slot cross 245 can incorporate aspects of the first pressure point mounting slot 220, the first pressure point mounting slot 230, the second pressure point mounting slot 225, and second pressure point mounting slot 235. First pressure point mounting slot cross 240 and second pressure point mounting slot cross 245 are oriented in a cross shape at substantially matching elevations on the backplate to provide vertically and horizontally adjustable locations for the pressure point knobs.

An alternative configuration of the backplate 105 with horizontal strap attachments is illustrated in FIG. 2D. It should be appreciated that all aspects of the backplate disclosed in other aspects of the backplate 105 (or backplate 505) can be incorporated in the backplate 105 with horizontal strap attachment. As illustrated in FIG. 2D, the backplate 105 can be fitted with a first horizontal mounting slot 250 and a second horizontal mounting slot 255.

In FIG. 2D the first horizontal mounting slot 250 and a second horizontal mounting slot 255 are illustrated. The horizontal mounting slots 250 and 255 are configured on the backplate 105 to be substantially in alignment at the two lengthwise ends of the backplate 105. This ensures the mounting slots serve as attachment points for the strap 120 for longitudinal mounting to a desired seat, backrest, backpack or the like.

Each of the horizontal mounting slots can include a mounting bar formed across the opening of the respective mounting slot. In FIG. 2D, mounting bar 251 is formed across horizontal mounting slot 250 and mounting bar 256 is formed across horizontal mounting slot 255. The horizontal mounting slots 250 and 255 and mounting bars 251 and 256 form strap mounting points through which the strap 120 can be threaded longitudinally.

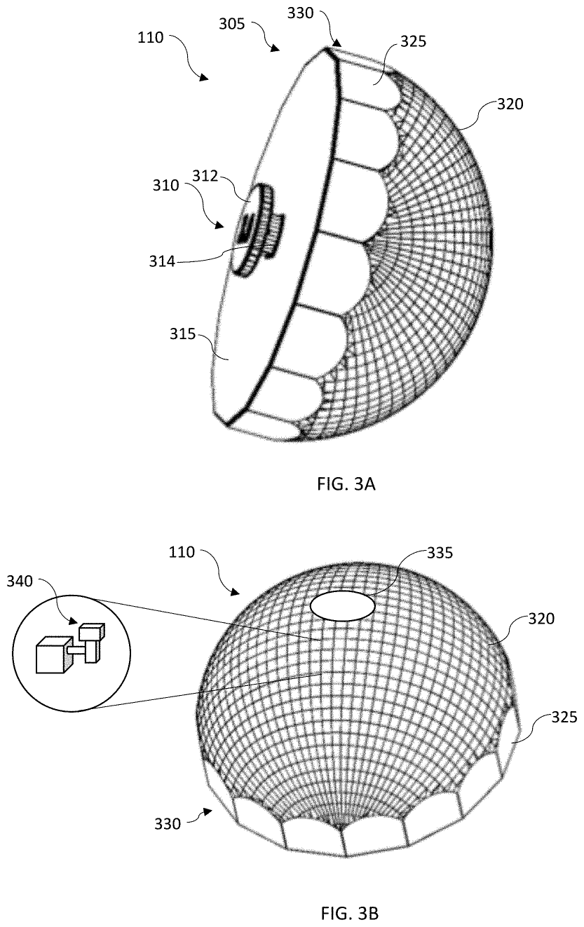

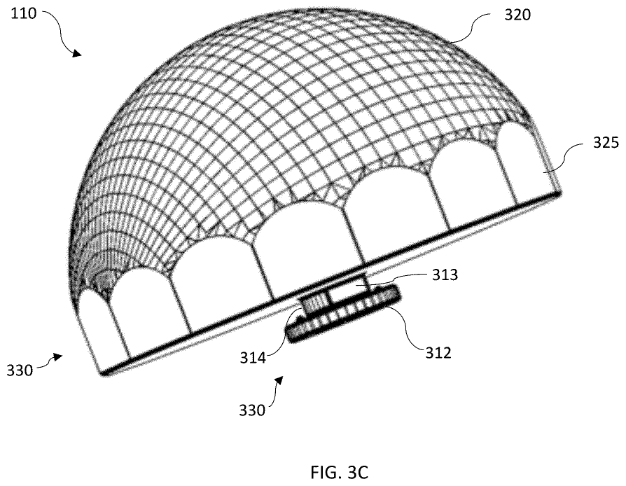

FIG. 3A illustrates first pressure point knob 110. It should be appreciated that FIG. 3 illustrates exemplary aspects of first pressure point knob 110 which can be included in second pressure point knob 115.

First pressure point knob 110 can generally comprise a knob body 305 and peg and post assembly 310. The knob body 305 serves as the interface that applies posture correcting and pain relieving pressure. The knob body 305 can include a flat bottom surface 315 and a rounded or domed outer surface 320. It should be appreciated that in other embodiments the shape of the outer surface 320 can be selected to be other shapes including but not limited to a pyramid type shape, bubbled shape, star shape, special characters or other such shape. The shape can be selected to maximize the posture corrective pressure and/or pain relief.

The rim 330 of the outer surface 320 can include a series of flattened panels 325, which facilitate grip on the knob body 305. In the embodiment illustrated in FIG. 3A panels 325 are butted against one another with the upper surface of each panel being an arc. This relationship creates a waved profile for the rim 330.

FIG. 3B illustrates another view of the first pressure point knob 110. From this view the top of the domed outer surface 320 is illustrated. In certain embodiments, an optional protuberance 335 can be fitted on the outer surface 320 of the knob body 305. This can comprise a simple pressure imparting protuberance. In other embodiments, the optional protuberance can comprise an electrode. As further detailed herein, the protuberance can be capable of delivering a desired amount of electrotherapy. FIG. 3B further illustrates a view of a vibrational motor 340 configured inside the first pressure point knob 110 as further detailed herein.

The peg and post assembly 310 includes a post 311 and a peg 312. The post 311 serves as the interface between the bottom surface 315 of the knob body 305 and the peg 312. The peg 312 can be configured to have a diameter that is slightly smaller than the diameter of the larger aperture 221 and/or larger aperture 226. Thus, the peg 312 can be inserted through the larger aperture in order to connect the pressure knob 110 to the backplate 105.

The post 311 can be specially designed with four surfaces, with two of the opposing surfaces being curved surfaces 314 and the other two opposing surfaces being flat surfaces 313. One of the curved surfaces 314 is illustrated in FIG. 3A. In FIG. 3C a flat surface 313 and an adjacent curved surface 314 are visible.

The shape of the post 311 is configured to interface with any of the disclosed pressure point mounting slots. In particular, the flat surfaces 313 are configured to fit in the mounting slots so that the pressure knob can slide along the pressure point mounting slot to a desired position. Once post reaches the desired position, the pressure point knob can be rotated 90 degrees along with the post 311 such that the curved surfaces 314 engage the smaller aperture cutout 222 to hold the pressure knob in place.

An aspect of the disclosed system 100 is the ability to fine tune the location of the pressure point knobs both with respect to one another, and with respect to the anatomy of the user to provide optimal posture correcting pressure and/or pain relief.

In certain embodiments, the pressure point knobs can further include a connection assembly between the post and peg assembly 310 and the knob body 305. Specifically, the distal end of the post 311 can include a threaded dowel configured to be screwed into a threaded opening formed in the knob body 305. In such an embodiment, the post and peg assembly 310 can be disengaged from the knob body 305. The knob body 305 can be positioned at a desired location along the pressure point mounting slot, and the post and peg assembly 310 can be reengaged to the knob body, holding the pressure point knob in place at the desired location.

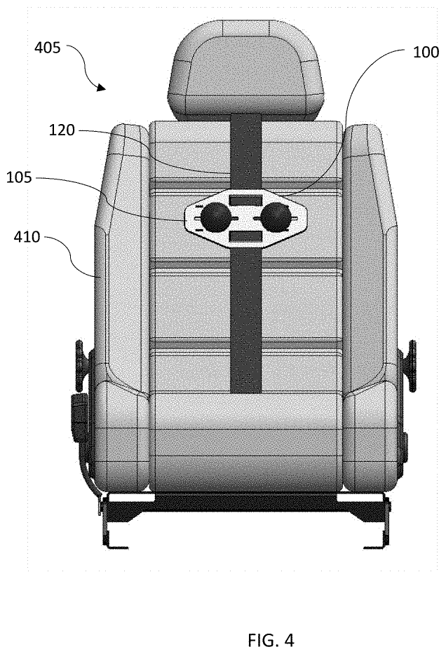

FIG. 4 illustrates an exemplary embodiment, wherein the posture corrective system 100 is mounted to a seat 405 in a vehicle. It should be appreciated that, in other embodiments, the posture corrective system 100 can be mounted to a chair of any kind, bench, back rest, pillow, backpack, or any other such device. As illustrated in FIG. 4, the strap 120 can be wrapped around the seatback 410 of the seat 405. The strap 120 can be secured in place by connecting the ends of the strap 120 with buckle 125. Tension in the strap 120 can be adjusted by pulling the tag end of the strap 120

Once the strap 120 is secured, the location of the backplate 105 can be adjusted with respect to the seat 405, to suit the user. This can include pulling slack through the mounting slots and sliding the backplate 105 along the strap 120 until the elevation of the backplate 105 on the seat back 410 is correctly positioned for the user. Likewise, the position of the pressure point knobs can be adjusted to suit the specific demands of the user.

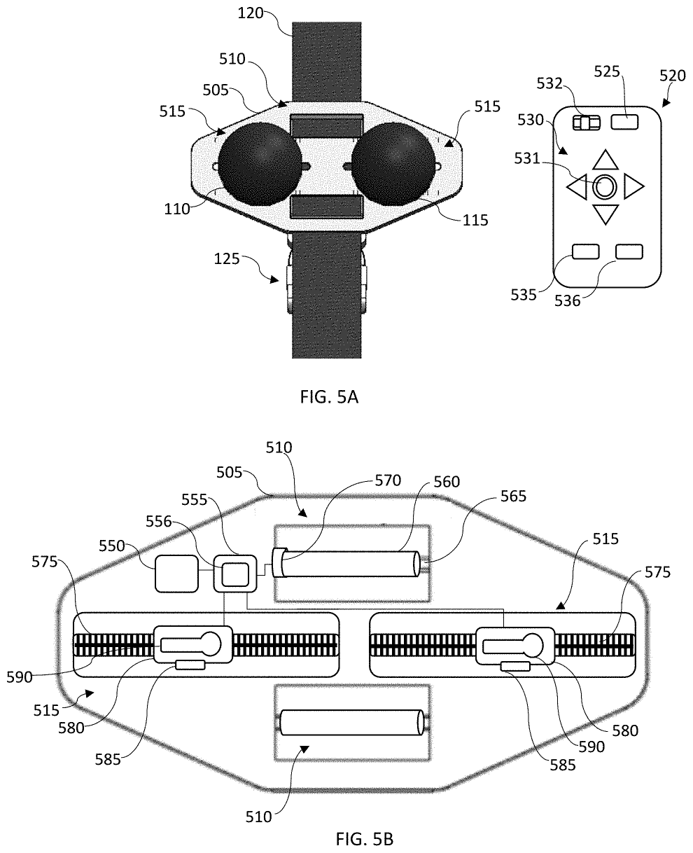

FIG. 5A illustrates another embodiment of a remotely controllable posture corrective system 500. The remotely controllable posture corrective system 500 can incorporate any of the features of other embodiments disclosed herein.

The remotely controllable posture corrective system 500, generally comprises a backplate 505, a first pressure point knob 110 and a second pressure point knob 115 which can be affixed to the backplate, and a strap 120 operably connected to the backplate. The strap 120 can have two loose ends each of which can be affixed to the male and female ends of a buckle 125, such that when the male and female ends of the buckle 125 are connected the strap forms a loop. The remotely controllable posture corrective system 500 further comprises at least one strap adjusting assembly 510, knob adjusting assembly 515, and remote control 520.

The remote control 520 can include a power button 525, as well as a directional pad 530. The buttons on the directional pad 530 can be used to control the location of the backplate 505 on the strap 120, via the strap adjusting assembly 510. The remote control 520 can further include a first pressure point knob selector switch 535 and a second pressure point knob selector switch 536. Pressing the pressure point knob selector switch will engage the appropriate knob adjusting assembly 515 so that the directional arrows on the directional pad 530 can be used to adjust the position of the associated pressure point knob. It should be appreciated that in certain embodiments, the remote control 520 can be integrated into the backplate 505, and/or the controls associated with the remote control 520 can be integrated into the backplate 505.

FIG. 5B illustrates aspects of the backplate 505 associated with the remotely controllable posture corrective system 500 in accordance with the disclosed embodiments. The backplate can include a power source 550, which can comprise a battery operably connected to a controller 555. The controller 555 can comprise an Arduino®, or other such microcontroller, and can be configured to receive wireless communication signals via transceiver 556. The controller 555 can accept wireless communication signals from the remote control 520.

The controller 555 can actuate the strap adjusting assembly 510 and/or knob adjusting assemblies 515 according to the instructions provided via wired or wirelessly from the remote control 520. In certain embodiments, the controller 555 can include wired connections to the strap adjusting assembly 510 and the knob adjusting assemblies 515, providing control signals and power to the respective assemblies.

In certain embodiments, the strap adjusting assembly 510 can include a roller 560 configured to rotate along an axel 565, with a rotational motor 570 connected to the axel and the controller 555. The rotational motor 570 can drive the axel, and in turn the roller 560. As the roller turns its outer surface engages the strap 120, thereby pulling the backplate 505 into position along the strap 120. It should be appreciated that the strap adjusting assembly disclosed herein is exemplary and other mechanisms for driving the strap can be used without departing from the scope of the disclosure herein. In certain embodiments, both mounting slots can have a strap adjusting assembly 510. In other embodiments, only one or the other of the mounting slots can have a strap adjusting assembly with the other mounting slot having a freely rotating roller on an axel, or a stationary mounting bar as disclosed in other embodiments.

In certain embodiments, the knob adjusting assembly or assemblies 515 can comprise a track 575 formed in the backplate 505. A mounting platform 580 can be operably engaged to the track 575. The mounting platform 580 can include a stepper motor 585 or other such motor device, configured to drive the mounting platform 580 along the track 575. The mounting platform 580 can include a gap mounting cutout 590, which allows a pressure point knob, such as first pressure point knob 110, or second pressure point knob 115, to be installed on the mounting platform 580. In other embodiments, the pressure point knob can be permanently mounted to the mounting platform 580.

In operation, the first pressure point knob selector switch 535 or the second pressure point knob selector switch 536 can be used to inform the controller which mounting platform 580 should be activated. The controller 555 can then use directional signals from the remote control 520 to operate the stepper motor 585 to automatically adjust the location of the mounting platform 580 along the track 575. Again, this description of the knob adjusting assembly 515 is meant to be exemplary and other mechanisms can be used in other embodiments.

It should be appreciated that aspects of the backplate 505 can be modified in accordance with configurations of backplate 105 to provide horizontal strap mounting, and/or two dimensional adjustments of the mounting platforms 580 with a cross style track configuration.

In further embodiments, the controller 555 and battery 550 can be further configured to control electrical nerve stimulating contacts. It should be appreciated that embodiments incorporating electrical nerve stimulating contacts can be realized in association with a posture corrective device 100 or a remotely controllable posture corrective system 500. In such embodiments, the controller 555 can be electrically connected to optional protuberance 335, which in certain embodiments, can comprise a nerve stimulating contact or transcutaneous electrical nerve stimulator.

The nerve stimulating contact can be a metal contact, associated with the pressure point knobs. In certain embodiments the nerve stimulating contact can comprise transcutaneous electrical nerve stimulator (TENS) or other such device. In order to create the necessary electrical connection between the pressure point knobs and the controller 555, in certain embodiments, the perimeter of the respective mounting slots (or gap mounting cutout) can be configured with an electrical contact. Similarly, the peg and post assembly 330 can comprise a metal contact so that when the peg and post assembly 330 engages the mounting slot or gap mounting cutout, an electrical connection is made. The peg and post assembly can further include electrical leads in contact with the nerve stimulating contacts 335. In other embodiments, where the pressure point knob is mounted directly to the backplate direct hardwired connections can be used.

The remote control 520 (or similar control provided on the backplate 100) can include stimulation hard button 531. The stimulation hard button 531 can be pressed to activate or deactivate electricity flow to the nerve stimulating contacts 335. The stimulation hard button 531 can further be configured as a dial, such that the strength of the nerve stimulation can be dialed higher or lower.

Additional vibrational massage can also be incorporated in the disclosed embodiments. In addition to the aspects disclosed above, in certain embodiments, the pressure knobs can be configured to be hollow. In such embodiments, the domed outer surface 320 can essentially form a shell connected to the rim 325. In such embodiments, a vibrational motor 340 can be configured inside the pressure knob 110. In certain embodiments the vibrational motor 340 can comprise an eccentric rotating mass on a dowel connected to a direct current motor. The direct current motor can turn the dowel and the mass connected thereto. The mass is unbalanced, so as it rotates it creates a vibrational effect. In other embodiments, other such vibrational motors 340 can be used. Electrical connections between the vibrational motor 340 and controller 555/battery 550 can be made as disclosed with respect to the nerve stimulating contact above.

The remote control 520 (or similar control provided on the backplate 100) can include toggle switch 532. The toggle switch 532 can be pressed to activate or deactivate electricity flow to the vibrational motor 340. The toggle switch 532 can further be configured as a slider, such that the strength of the vibration can be dialed higher or lower.

In certain embodiments, the strap 120 of the posture corrective system 100 can comprise a textile strap, elastic strap, cloth strap, rubber strap, or other such strap. A buckle 125, which can alternatively be a clasp, snaps, buttons, hook and loop fasteners, or other such connecting means, can be configured to engage the first end of the strap 120 with the second end of the strap 120. The strap 120 can be used to removably mount the posture corrective system 100 to a seat.

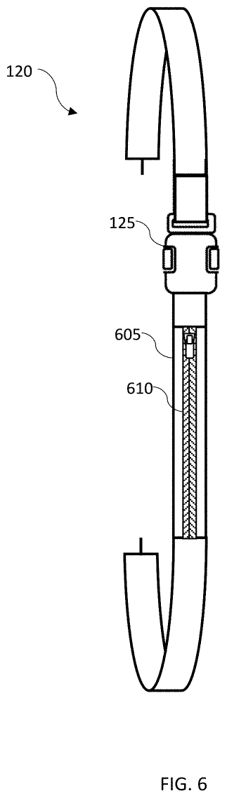

FIG. 6 illustrates additional aspects of the strap 120. In some circumstances, a chair, seat or other such circumstances where the seat can't easily accommodate the strap may arise. In such embodiments, the strap 120 can include a section 605 fitted with a zipper 610. The section of the strap 605 can be separated by opening the zipper 610. In this way, the section 605 can fit around the spine of the chair, or other such obstruction, so that the system 100 or system 500 can easily mount to the chair.

In accordance with the disclosure herein, the posture correction device can be used in association with automobile seats, office chairs of any kind, backpacks of any kind, or other such seats with back rests. The two pressure points that sit at the center of the backplate can be adjusted to specifications of the user. The adjustable strap runs through the backplate and can wrap around a chair, backpack, or pillow. The backplate can slide up and down the strap and be positioned taut at the spinal region that is most comfortable for the wearer, and can be adjusted to any region of the spine (Cervical, Thoracic or Lumbar region) by simply moving the backplate upwards or downwards along the adjustable strap.

Based on the foregoing, it can be appreciated that a number of embodiments, preferred and alternative, are disclosed herein. For example, in an embodiment a posture corrective system comprises a backplate, at least two pressure point knobs affixed to the backplate, and a strap operably connected to the backplate.

In an embodiment, the system comprises at least one mounting slot formed in the backplate and at least one mounting bar bisecting the mounting slot, the mounting slot and mounting bar forming a strap mounting point through which the strap can be threaded.

In an embodiment, the system comprises at least two pressure point mounting slots formed on the backplate, wherein each of the at least two pressure point knobs can be mounted to the backplate via the at least two pressure point mounting slots. In certain embodiments, the at least two pressure point mounting slots comprise straight slots configured at a substantially matching elevation on the backplate. In certain embodiments, the at least two pressure point mounting slots comprise straight slots are oriented to be parallel and are configured at a substantially matching elevation on the backplate. In certain embodiments, the at least two pressure point mounting slots comprise cross shaped slots and are configured at a substantially matching elevation on the backplate.

In an embodiment, each of the at least two pressure point knobs further comprise threaded mounting holes configured to accept a screw. In another embodiment, each of the at least two pressure point knobs further comprise a peg and post assembly.

In an embodiment, the system comprises a clasp configured to engage a first end of the strap with a second end of the strap.

In an embodiment, the system comprises a battery and at least one electrode operably connected to the battery and configured on at least one of the at least two pressure point knobs.

In another embodiment a posture corrective system comprises a backplate, at least two pressure point knobs affixed to the backplate, at least one controller formed on the backplate configured to control a location of the at least two pressure point knobs, at least one battery, and a strap operably connected to the backplate.

In an embodiment, the system comprises at least one strap adjusting assembly operably connected to the at least one controller and configured to adjust a location of the backplate on the strap. In an embodiment, the system comprises at least two knob adjusting assemblies operably connected to the at least one controller and configured to adjust the respective location of the at least two pressure point knobs.

In an embodiment, the system comprises a transceiver associated with the controller and a remote control configured to provide instructions to the controller.

In an embodiment, the system comprises at least one electrode operably connected to the battery and the controller and configured on at least one of the at least two pressure point knobs. In an embodiment, the system comprises at least one vibrational motor operably connected to the battery and the controller and configured in at least one of the at least two pressure point knobs.

In another an embodiment, the apparatus comprises a posture corrective apparatus comprising a backplate, at least two pressure point knobs affixed to the backplate, a strap operably connected to the backplate and a zipper configured along a portion of the strap.

In an embodiment, the apparatus comprises at least one mounting slot formed in the backplate and at least one mounting bar bisecting the mounting slot, the mounting slot and mounting bar forming a strap mounting point through which the strap can be threaded. In an embodiment, at least two pressure point mounting slots formed on the backplate, wherein each of the at least two pressure point knobs can be mounted to the backplate via the at least two pressure point mounting slots. In certain embodiments, each of the at least two pressure point knobs further comprise a protuberance formed on an outer surface of each of the at least two pressure point knobs.

It will be appreciated that variations of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art which are also intended to be encompassed by the following claims.

What is claimed is:

1. A posture corrective system comprising:
a backplate;
at least two pressure point knobs affixed to the backplate; and
a strap operably connected to the backplate.

2. The posture corrective system of claim 1 further comprising:
at least one mounting slot formed in the backplate; and
at least one mounting bar bisecting the mounting slot, the mounting slot and mounting bar forming a strap mounting point through which the strap can be threaded.

3. The posture corrective system of claim 1 further comprising:
at least two pressure point mounting slots formed on the backplate, wherein each of the at least two pressure point knobs can be mounted to the backplate via the at least two pressure point mounting slots.

4. The posture corrective system of claim 3 wherein the at least two pressure point mounting slots comprise straight slots configured at a substantially matching elevation on the backplate.

5. The posture corrective system of claim 3 wherein the at least two pressure point mounting slots comprise straight slots oriented to be parallel and configured at a substantially matching elevation on the backplate.

6. The posture corrective system of claim 3 wherein the at least two pressure point mounting slots comprise cross shaped slots and are configured at a substantially matching elevation on the backplate.

7. The posture corrective system of claim 1 wherein each of the at least two pressure point knobs further comprise:
threaded mounting holes configured to accept a screw.

8. The posture corrective system of claim 1 wherein each of the at least two pressure point knobs further comprise:
a peg and post assembly.

9. The posture corrective system of claim 1 further comprising:
a clasp configured to engage a first end of the strap with a second end of the strap.

10. The posture corrective system of claim 1 further comprising:
a battery; and
at least one electrode operably connected to the battery and configured on at least one of the at least two pressure point knobs.

11. A posture corrective system comprising:
a backplate;
at least two pressure point knobs affixed to the backplate;
at least one controller formed on the backplate configured to control a location of the at least two pressure point knobs;
at least one battery; and
a strap operably connected to the backplate.

12. The posture corrective system of claim 11 further comprising:
at least one strap adjusting assembly operably connected to the at least one controller and configured to adjust a location of the backplate on the strap.

13. The posture corrective system of claim 11 further comprising:
at least two knob adjusting assemblies operably connected to the at least one controller and configured to adjust the respective location of the at least two pressure point knobs.

14. The posture corrective system of claim 11 further comprising:
a transceiver associated with the controller; and
a remote control configured to provide instructions to the controller.

15. The posture corrective system of claim 11 further comprising:
at least one transcutaneous electrical nerve stimulator operably connected to the battery and the controller and configured on at least one of the at least two pressure point knobs.

16. The posture corrective system of claim 11 further comprising:
at least one vibrational motor operably connected to the battery and the controller and configured in at least one of the at least two pressure point knobs.

17. A posture corrective apparatus comprising:
a backplate;
at least two pressure point knobs affixed to the backplate;
a strap operably connected to the backplate and a zipper configured along a portion of the strap.

18. The posture corrective apparatus of claim 17 further comprising:
at least one mounting slot formed in the backplate; and
at least one mounting bar bisecting the mounting slot, the mounting slot and mounting bar forming a strap mounting point through which the strap can be threaded.

19. The posture corrective system of claim 1 further comprising:
at least two pressure point mounting slots formed on the backplate, wherein each of the at least two pressure point knobs can be mounted to the backplate via the at least two pressure point mounting slots.

20. The posture corrective system of claim 1 wherein each of the at least two pressure point knobs further comprise:
a protuberance formed on an outer surface of each of the at least two pressure point knobs.
